(19) United States
(12) Patent Application Publication
(10) Pub. No.: US 2025/0308626 A1
(43) Pub. Date: Oct. 2, 2025

(54) METHOD FOR MOLECULAR DOCKING AND ELECTRONIC DEVICE

(71) Applicants: Beijing Youzhuju Network Technology Co., Ltd., Beijing (CN); Lemon Inc., Cayman Islands (KY)

(72) Inventors: Yiqun WANG, Los Angeles, CA (US); Lihao WANG, Beijing (CN); Yuning SHEN, Los Angeles, CA (US); Shi CHEN, Los Angeles, CA (US)

(21) Appl. No.: 18/864,470

(22) PCT Filed: Aug. 28, 2023
(86) PCT No.: PCT/CN2023/115311

§ 371 Date: Nov. 8, 2024

(30) Foreign Application Priority Data
Sep. 21, 2022 (CN) 202211151228.1

(51) Int. Cl. G16B 15/30; G06N 3/086

(52) U.S. Cl. CPC G16B 15/30; G06N 3/086

(57) ABSTRACT

Embodiments of the present disclosure relate to a method for molecular docking and an electronic device. The method comprises: determining a first binding site on a first molecular surface of a first molecule and a second binding site on a second molecular surface of a second molecule based on a first time-dependent evolution multiscale feature of the first molecule and a second time-dependent evolution multiscale feature of the second molecule; obtaining a first chemical feature of the first binding site and a second chemical feature of the second binding site; determining a functional mapping matrix between the first chemical feature and the second chemical feature through functional mapping; determining a correspondence between the first binding site and the second binding site based on the functional mapping matrix; and docking the first molecule and the second molecule through the first binding site and the second binding site based on the correspondence.

### CROSS-REFERENCE TO RELATED APPLICATION(S)

The present application claims priority to Chinese Patent Application No. 202211151228.1, filed with the China National Intellectual Property Administration on Sep. 21, 2022 and entitled “METHOD FOR MOLECULAR DOCKING AND ELECTRONIC DEVICE”, which is incorporated herein by reference in its entirety.

### FIELD

The present disclosure generally relates to the field of computers and the field of bioinformatics, and more particularly to a method for molecular docking and an electronic device.

### BACKGROUND

The interaction between biomolecules is an important basis for achieving their biological activities. For example, the human body can generate antibody proteins that bind to invading viruses to inhibit diseases. In biopharmaceutical research, it is possible to understand the physical and chemical mechanisms of intermolecular interactions by analyzing those biomolecules that are known to bind to each other, thereby helping to design novel drug molecules that can bind to some specific targets (such as developing a new coronavirus antibody). In this process, molecular docking is an important research direction.

One of the existing solutions is to determine possible binding sites for molecular docking through massive sampling, and then dock the molecules. However, such a solution is costly and time-consuming, resulting in low efficiency of molecular docking.

### SUMMARY

According to example embodiments of the present disclosure, a method for molecular docking is provided, in which a binding site is determined based on a time-dependent evolution multiscale feature, and molecular docking is achieved through functional mapping.

In a first aspect of embodiments of the present disclosure, a method for molecular docking is provided, including: determining a first binding site on a first molecular surface of a first molecule and a second binding site on a second molecular surface of a second molecule based on a first time-dependent evolution multiscale feature of the first molecule and a second time-dependent evolution multiscale feature of the second molecule; obtaining a first chemical feature of the first binding site and a second chemical feature of the second binding site; determining a functional mapping matrix between the first chemical feature and the second chemical feature through functional mapping; determining a correspondence between the first binding site and the second binding site based on the functional mapping matrix; and docking the first molecule and the second molecule through the first binding site and the second binding site based on the correspondence.

In a second aspect of embodiments of the present disclosure, an electronic device is provided, including: at least one processing unit; and at least one memory coupled to the at least one processing unit and storing instructions executable by the at least one processing unit, the instructions, when executed by the at least one processing unit, causing the electronic device to perform the method described in accordance with the first aspect of the present disclosure.

In a third aspect of embodiments of the present disclosure, a computer-readable storage medium is provided, having machine-executable instructions stored thereon, the machine-executable instructions, when executed by a device, causing the device to perform the method described in accordance with the first aspect of the present disclosure.

In a fourth aspect of embodiments of the present disclosure, a computer program product is provided, including computer-executable instructions, where the computer-executable instructions, when executed by a processor, implement the method described in accordance with the first aspect of the present disclosure.

In a fifth aspect of embodiments of the present disclosure, an electronic device is provided, including: a processing circuit configured to perform the method described in accordance with the first aspect of the present disclosure.

The Summary section is provided to introduce a series of concepts in a simplified form, which will be further described below in the Detailed Description. The Summary section is not intended to identify key features or essential features of the present disclosure, nor is it intended to limit the scope of the present disclosure. Other features of the present disclosure will become readily understandable through the following description.

### BRIEF DESCRIPTION OF THE DRAWINGS

The above and other features, advantages, and aspects of embodiments of the present disclosure become more apparent with reference to the following detailed description and in conjunction with the accompanying drawings. In the drawings, the same or similar reference numerals denote the same or similar elements, where:

FIG. 1 illustrates a schematic flowchart of an example process in accordance with some embodiments of the present disclosure;

FIG. 2A illustrates a schematic diagram of an electron density field of a benzene molecule in accordance with some embodiments of the present disclosure;

FIG. 2B illustrates a schematic diagram of a molecular surface represented by triangulation in accordance with some embodiments of the present disclosure;

FIG. 3A illustrates a schematic diagram of projecting chemical information of atoms to a node of a molecular surface in accordance with some embodiments of the present disclosure;

FIG. 3B illustrates a schematic diagram of an electrostatic potential energy function of a molecular surface in accordance with some embodiments of the present disclosure;

FIG. 4 illustrates a schematic diagram of the distribution of the first six eigenfunctions of a molecule on a molecular surface in accordance with some embodiments of the present disclosure;

FIG. 5 illustrates a schematic diagram of a change in heat distribution on a molecular surface over time in accordance with some embodiments of the present disclosure;

FIG. 6 illustrates a schematic diagram of using a cross-attention network in accordance with some embodiments of the present disclosure;

FIG. 7 illustrates a schematic diagram of molecular docking in accordance with some embodiments of the present disclosure;

FIG. 8 illustrates a block diagram of an example apparatus in accordance with some embodiments of the present disclosure; and

FIG. 9 illustrates a block diagram of an example device that can be used to implement embodiments of the present disclosure.

### DETAILED DESCRIPTION OF EMBODIMENTS

Embodiments of the present disclosure will be described in more detail below with reference to the accompanying drawings. Although some embodiments of the present disclosure are shown in the drawings, it should be understood that the present disclosure may be implemented in various forms and should not be construed as being limited to embodiments set forth herein. Rather, these embodiments are provided for a more thorough and complete understanding of the present disclosure. It should be understood that the drawings and embodiments of the present disclosure are only for exemplary purposes, and are not intended to limit the scope of protection of the present disclosure.

As mentioned above, molecular docking is an important direction in the field of biomolecular research. For example, molecular docking can be implemented through computer modeling to simulate how two molecules interact and combine in a real organism.

Taking a pair of receptor protein and ligand protein as an example, the physicochemical properties and geometric structure of the receptor protein and the ligand protein can be analyzed, and the ligand protein can be bound to the binding site of the receptor protein. Through docking, it is possible to predict the three-dimensional structure of the complex formed by binding of the receptor protein and the ligand protein. However, the current solutions cannot efficiently achieve the docking between two molecules.

At least to solve the above problems and other potential problems, embodiments of the present disclosure provide a solution for molecular docking. Specifically, the binding sites may be determined based on the respective time-dependent evolution multiscale features of the two molecules, and then molecular docking may be further implemented through functional mapping based on the chemical features of the binding sites. This solution does not need to be implemented through a large number of samplings, so that the three-dimensional structure formed after docking can be determined more quickly and more efficiently.

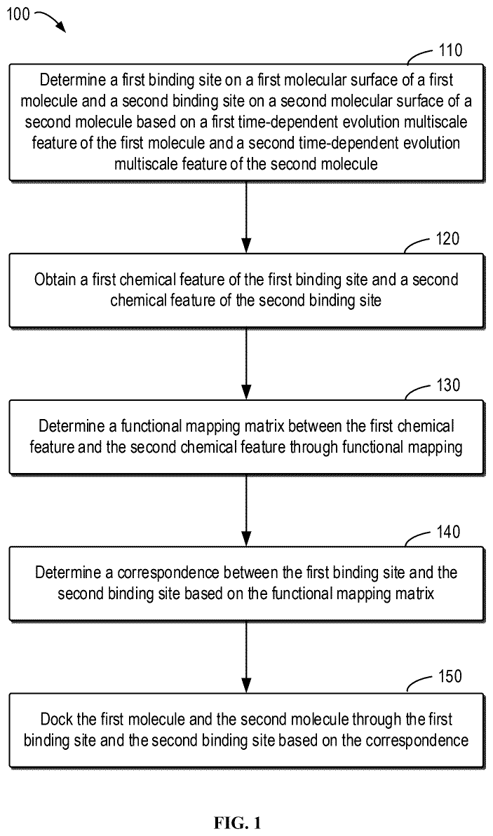

FIG. 1 illustrates a schematic flowchart of an example process 100 in accordance with some embodiments of the present disclosure. At block 110, a first binding site on a first molecular surface of a first molecule and a second binding site on a second molecular surface of a second molecule are determined based on a first time-dependent evolution multiscale feature of the first molecule and a second time-dependent evolution multiscale feature of the second molecule. At block 120, a first chemical feature of the first binding site and a second chemical feature of the second binding site are obtained. At block 130, a functional mapping matrix between the first chemical feature and the second chemical feature is determined through functional mapping. At block 140, a correspondence between the first binding site and the second binding site is determined based on the functional mapping matrix. At block 150, the first molecule and the second molecule are docked through the first binding site and the second binding site based on the correspondence.

Exemplarily, the molecules (such as the first molecule and the second molecule) in embodiments of the present disclosure may be biological macromolecules, such as proteins, DNA, and the like; or may be small molecules, such as aspirin drug small molecules. The present disclosure is not limited thereto. For the purpose of a simplified schematic illustration, some of the following embodiments are described by taking proteins as an example.

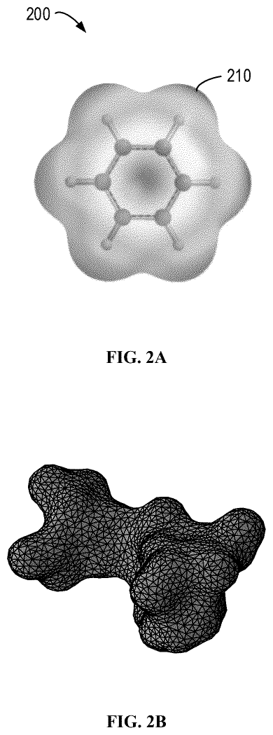

In some embodiments, it may be understood that before block 110, the first time-dependent evolution multiscale feature of the first molecule and the second time-dependent evolution multiscale feature of the second molecule may be determined respectively. In embodiments of the present disclosure, the process of determining the first time-dependent evolution multiscale feature is similar to the process of determining the second time-dependent evolution multiscale feature. The process of determining a time-dependent evolution multiscale feature of any molecule will be described in conjunction with FIGS. 2A-5 below. It may be understood that the first time-dependent evolution multiscale feature may be determined through a similar process, and the second time-dependent evolution multiscale feature of the second molecule may also be determined through a similar process.

Exemplarily, for any molecule, a molecular surface of the molecule may be determined, where the molecular surface is a continuous Riemannian manifold and the molecular surface includes a plurality of discrete surface nodes; a geometric feature of the molecule is determined based on the molecular surface; a surface chemical feature of the molecule is determined by mapping atomic information inside the molecule to the plurality of surface nodes; and a time-dependent evolution multiscale feature of the molecule is determined based on the geometric feature and the surface chemical feature.

In some exemplary embodiments of the present disclosure, the molecular surface of the molecule may be determined based on an isosurface of an electron density field of the molecule.

The scale of biomolecules is generally in units of 10⁻¹⁰ meters (angstroms). At this microscopic scale, biomolecules generally follow the physical laws described by quantum mechanics and statistical mechanics, rather than Newtonian mechanics at the macroscopic scale. From the perspective of microscopic electronic structure, a molecule consists of a positively charged atomic nucleus and a negatively charged electron cloud. Intuitively, a molecule can be understood as an electron density field. Different biomolecules have different chemical composition and three-dimensional geometric structures, thereby showing different physicochemical properties. For example, a specific drug molecule will bind to a certain protein receptor in the human body to achieve therapeutic effect. In other words, different molecules have their unique electron density fields, and thus different molecules can be represented by describing the shape and chemical properties of the density fields. Specifically, an isosurface of the density field may be determined, which is referred to as a molecular surface of the molecule.

As an example, FIG. 2A illustrates an electron density field 200 of a benzene molecule in accordance with embodiments of the present disclosure. In FIG. 2, a curve 210 represents an isosurface.

Exemplarily, the electron density field of a molecule may be represented as an electron density function of the molecule. Optionally, the electron density function of the molecule may be determined through quantum chemical simulation, and further, the molecular surface may be determined based on an isosurface of the electron density function of the molecule. For example, there may be a plurality of isosurfaces for the electron density function of the molecule, and thus in some embodiments of the present disclosure, the molecular surface may be determined by selecting one of the isosurfaces.

In some exemplary embodiments of the present disclosure, the molecular surface may also be determined through other molecular surface calculation methods. For example, the molecular surface of the molecule may be determined by using MSMS calculation software.

In some exemplary embodiments of the present disclosure, the molecular surface of the molecule may also be determined based on sampling of the solvent-accessible or solvent-inaccessible surfaces of the molecule.

It may be understood that in some other examples, the molecular surface of the molecule may also be determined in other manners in embodiments of the present disclosure, which is not limited in the present disclosure.

In some examples, the molecular surface may be represented as a plurality of discrete nodes and connection relationships between the nodes. Exemplarily, surface information may be further determined based on the determined molecular surface. For example, the surface information may be stored by using a mesh representation method such as triangulation. FIG. 2B illustrates a schematic diagram of a molecular surface represented by triangulation. As illustrated, there are triangulation nodes (referred to as “nodes” for short) shown on the surface, and there may be connection relationships between the nodes. In other words, the molecular surface includes a plurality of surface nodes, e.g., a plurality of triangulation nodes.

Exemplarily, the surface wraps the molecule and can express the shape of the molecule. In embodiments of the present disclosure, the stored surface information may include: atomic information inside the molecule, and three-dimensional coordinates of each node and connection relationships between the nodes on the molecular surface. For example, the atomic information inside the molecule includes related chemical information such as three-dimensional coordinates and an atom type of the atom. It may be understood that the molecular surface is a two-dimensional Riemannian manifold, and the manifold itself is continuous and smooth. In the subsequent processing of embodiments of the present disclosure, the continuous and smooth Riemannian manifold may be discretized to, e.g., triangulation nodes.

In some exemplary embodiments of the present disclosure, for each node in the plurality of surface nodes, a chemical environment feature of the node is obtained by mapping atomic information of a plurality of atoms associated with the node to the node; and the chemical feature is determined using a fully connected neural network based on the chemical environment feature of each node in the plurality of surface nodes. Exemplarily, the plurality of atoms associated with the node may include: a plurality of atoms within a range where the distance to the node is lower than a distance threshold. Alternatively, exemplarily, the plurality of atoms associated with the node include: a fixed number of nearest neighbor atoms (for example, 8 nearest neighbor atoms) that are closest to the node. For example, the atoms may be sorted according to the distance to the node, and the nearest fixed number (such as 8) of atoms may be determined from the sorted atoms.

Specifically, a chemical potential distribution of the molecular surface may be determined based on the surface information of the molecule. Optionally, the chemical potential distribution may also be referred to as a chemical function distribution, e.g., an electrostatic potential energy distribution.

Exemplarily, for any node on the molecule surface, the distance between all atoms within a specific distance range around the node and the node may be determined. For example, atoms within the distance threshold range may be referred to as neighbor atoms. Subsequently, the normal angle between each neighbor atom and a tangent plane of the curved surface where the node is located and the corresponding atom type may be determined, and used as the initial representation of the chemical environment of the node. Exemplarily, the chemical function distribution of the molecular surface may be extracted through a fully connected neural network. In other words, the representation of the surrounding chemical environment of the surface node can be learned through the fully connected neural network.

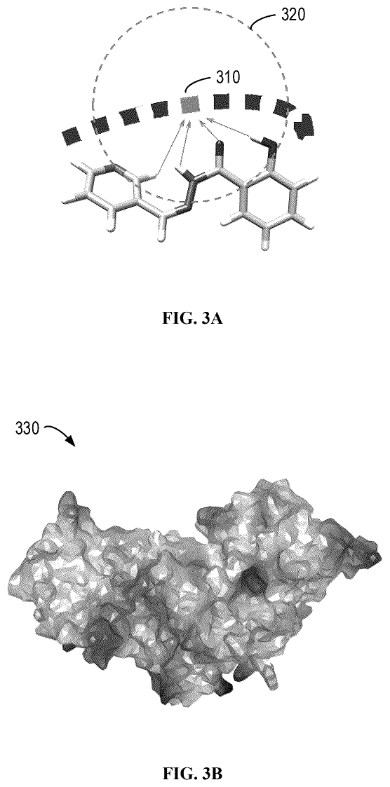

In this way, by mapping (also referred to as projection) the chemical information of the internal atoms to the surface nodes, the chemical information of the entire molecule can be characterized by the nodes of the molecular surface. FIG. 3A illustrates a schematic diagram of projecting chemical information of atoms to a node of a molecular surface. As shown in FIG. 3A, for a node 310, atoms within a specific distance range 320 may be determined. Subsequently, the chemical information of the determined atoms may be projected onto the node 310 to determine the initial representation of the chemical environment of the node 310, such as the chemical environment feature of the node.

It should be noted that in embodiments of the present disclosure, the chemical representation of the node on the molecular surface may be updated by using the chemical information of atoms, but the information of the node will not feedback and change the chemical information of atoms, that is, the projection belongs to a one-way information transfer relationship, which is different from a graph neural network of a molecule with two-way update. It may be understood that although the graph neural network can realize long-distance information exchange through graph information transfer, the exchange mechanism is inefficient when there are a large number of nodes (for example, there are usually tens of thousands of nodes in the surface triangulation representation of a molecule). In contrast, in embodiments of the present disclosure, the processing efficiency of information exchange can be improved through the one-way information transfer relationship from the atomic information to the node.

Exemplarily, through the fully connected neural network, the chemical feature of the molecular surface may be determined based on the chemical environment feature of each of the plurality of surface nodes. Optionally, as an example, the chemical information of an atom may be represented as a multi-dimensional (such as 5-dimensional) array, and the surface chemical feature may be represented as a multi-dimensional (such as 16-dimensional) array.

FIG. 3B illustrates a schematic diagram of an electrostatic potential energy function 330 of a molecular surface. For example, the electrostatic potential energy function may be obtained by extracting the first-dimensional feature from the chemical feature of, e.g., a 16-dimensional array. It may be understood that although FIG. 3B is described by taking the electrostatic potential energy function as an example, embodiments of the present disclosure are not limited thereto. For example, a user may customize other chemical information, or may learn other chemical representations through a neural network or the like.

In this way, the chemical potential distribution of the molecular surface may include both geometric information and chemical information. Exemplarily, the distribution of the chemical potential function such as the electrostatic potential energy function on the molecular surface belongs to the surface Riemannian manifold space representation of the molecule, that is, the chemical information may exist in the form of a function in the surface Riemannian manifold space of the molecule. In other words, in embodiments of the present disclosure, the surface of the molecule is regarded as a continuous and smooth Riemannian manifold space, and a chemical-related function is defined in the two-dimensional manifold space.

In some exemplary embodiments of the present disclosure, the geometric feature may include one or more of the following: a heat kernel signature, a wave kernel signature, Gaussian curvature of the molecular surface, or mean curvature of the molecular surface.

Exemplarily, the eigenfunction of the Laplace operator on the molecular surface (or referred to as the Laplace eigenfunction for short) and the eigenvalue may be determined, and the heat kernel signature and/or the wave kernel signature are determined based on the eigenfunction and the eigenvalue.

Exemplarily, the eigenfunction and the eigenvalue of the Laplace operator (Laplace-Beltrami operator) on each molecular surface Riemannian manifold may be determined, which is expressed as the following formula (1):

$${Δϕ}_{i} = {λ}_{i} {ϕ}_{i} \qquad ( 1 )$$

In formula (1), Δ represents the Laplace operator, and its meaning is shown in the following formula (2):

$$Δ f = {∇}^{2} f = ∇ \cdot ∇ f = \frac{{\partial }^{2} f}{\partial {x}^{2}} + \frac{{\partial }^{2} f}{\partial {y}^{2}} \qquad ( 2 )$$

In formula (1), Øᵢ represents the ith eigenfunction, and λᵢ represents the ith eigenvalue. In formula (2), □ represents the gradient operator, and ƒ represents any function distributed on the Riemannian manifold. Exemplarily, the eigenfunction may be determined by using a known (for example, scipy numerical calculation software) or a future-developed algorithm, etc., which is not limited in the present disclosure.

In some examples, the Laplace eigenfunction of each molecular surface manifold and its corresponding eigenvalue are unique, and only related to the shape of the molecule itself, and not affected by the position and orientation of the molecule in the three-dimensional space. Therefore, the eigenfunction of the Riemannian manifold is also referred to as “shape DNA”. For the surface manifold of each molecule, all its eigenfunctions and eigenvalues may be determined. Exemplarily, the eigenvalues may be further sorted according to the size of the eigenvalues, for example, the eigenvalues may be sorted in an ascending order, and then the first k eigenvalues (for example, k=100 or other values) in the sort are taken, in this way, the amount of calculation can be reduced.

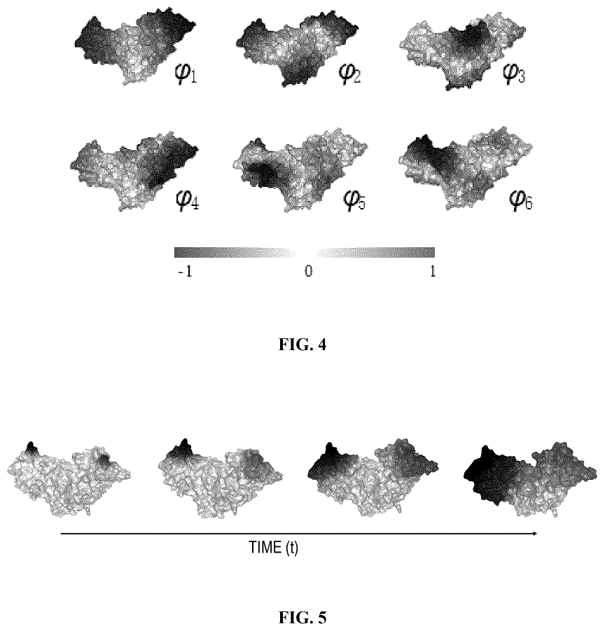

It may be understood that since different biomolecules have different shapes, there are also different surface manifold eigenfunctions. FIG. 4 illustrates the distribution of the first six eigenfunctions of a molecule on the molecular surface in accordance with some embodiments of the present disclosure. Exemplarily, the first six eigenfunctions are shown as φ₁-φ₆ in FIG. 4. In some examples, the eigenfunction shows regional undulation in FIG. 4, and accordingly, the eigenfunction may be understood as a Fourier basis function (for example, may be understood as a two-dimensional standing wave) in the two-dimensional manifold space, which corresponds to a sine function and a cosine function on the one-dimensional straight line.

In some exemplary embodiments of the present disclosure, the geometric feature may be represented in the form of a geometric feature function. The geometric feature function of the molecular surface may be determined based on the eigenfunction and the eigenvalue of the Laplace operator on the molecular surface manifold. Optionally, the geometric feature function may include a heat kernel signature (HKS) and/or a wave kernel signature (WKS).

Exemplarily, the HKS and the WKS may be constructed based on the determined eigenfunction Øᵢ and the eigenvalue λᵢ as follows:

$$HKS ( x , t ) = {\sum }_{i} {e}^{- {λ}_{i} t} {ϕ}_{i}^{2} ( x ) \qquad ( 3 )$$

$$WKS ( x , ϵ ) = {\sum }_{k} {ϕ}_{k}^{2} ( x ) {e}^{\frac{- {( ϵ - l og {E}_{k} )}^{2}}{2 {σ}^{2}}} \qquad ( 4 )$$

In formulas (3) and (4), t and ∈ represent time and energy respectively, which may be set by the user, for example.

Optionally, the geometric feature function of the molecular surface may also include Gaussian curvature and/or mean curvature on the molecular surface (Riemannian manifold). It may be understood that the Gaussian curvature and the mean curvature may be calculated through a geometric method, which will not be repeated here.

In some exemplary embodiments of the present disclosure, the unified feature of the molecule may be determined by integrating the geometric feature and the chemical feature. For example, the geometric feature is represented as a geometric feature function, and the chemical feature is represented as a chemical potential distribution, then the unified feature of the molecular surface may be determined based on the chemical potential distribution of the molecular surface and the geometric feature function of the molecular surface. The unified feature (for example, represented as a surface feature function) may represent the integration of chemical information and geometric information.

Exemplarily, the chemical feature and the geometric feature of each node may be integrated through a fully connected neural network, to obtain the surface feature function at each node. For example, assuming that the chemical feature is represented as a 16-dimensional array, and the geometric feature is represented as a 32-dimensional array, the chemical feature and the geometric feature may be nonlinearly transformed into a 64-dimensional surface feature function through the fully connected neural network. It may be understood that the dimension of the surface feature function is not limited to 64 dimensions, and it may be customized by the user, such as 128 dimensions or other dimensions, which is not limited in the present disclosure.

Exemplarily, the fully connected neural network may be obtained through training based on a molecular data set. Specifically, the molecular data set is related to the application scenario of embodiments of the present disclosure (for example, a downstream prediction task).

In some exemplary embodiments of the present disclosure, the time-dependent evolution multiscale feature may be determined based on the unified feature using a time-dependent evolution neural network model. Exemplarily, the time-dependent evolution multiscale feature represents a multiscale feature of the molecular surface.

Exemplarily, the time-dependent evolution neural network model includes an evolution operator, and the evolution operator is at least based on the Laplace operator and/or based on a surface potential energy term.

For example, the time-dependent evolution operator may be applied to the surface feature function to obtain a function representing the multiscale feature. For example, the time-dependent evolution operator may be represented as e^{-iĤt} or e^{-Ĥt}, where Ĥ is a Hamiltonian operator, for example, Ĥ=Δ+V. Δ represents the Laplace operator, and V represents the surface potential energy term. For example, the surface potential energy term V may be a function distribution on the manifold set by the user.

In some embodiments, when the time-dependent evolution operator is represented as e^{-iĤt} for an initial function u₀, the function distribution at time t may be determined by the following formula (5):

$${u}_{t} = {e}^{- i \hat{H} t} {u}_{0} \qquad ( 5 )$$

For the purpose of a simplified example, it may be assumed that V=0, so that formula (5) can be simplified to the following formula (6):

$${u}_{t} = {e}^{- i Δ t} {u}_{0} \qquad ( 6 )$$

Formula (6) describes the change of an initial function u₀ over time in the manifold space (that is, the molecular surface). By controlling different evolution times t, a new function distribution uₜ after evolution at different times can be obtained. It may be understood that uₜ obtained by formula (6) is a complex number, while the input u₀ is a real number. In practice, the modulus of uₜ may be taken to obtain a real number corresponding to uₜ.

Since different molecules have different geometric structures, their Riemannian manifold spaces are also unique, and the evolution mode of the function u₀ on different manifolds is also determined by the manifold space. Therefore, the evolved function can be used as a new representation of molecular information, and this representation contains the global and local information of the manifold.

In some other embodiments, when the time-dependent evolution operator is represented as e^{-Ĥt} and V=0, for an initial function v₀, the function distribution at time/may be determined by the following formula (7):

$${t}_{} = {e}^{- Δ t} {0}_{} \qquad ( 7 )$$

Formula (7) may be understood as replacing the imaginary time-dependent evolution operator in the above formula (6) with a real time-dependent evolution operator (removing i). It may be understood that formula (6) belongs to a quantum mechanical framework, and formula (7) belongs to a classical mechanical framework. In practical applications, both of these two frameworks can be used to implement the Riemannian manifold representation of the molecule.

In embodiments of the present disclosure, the initial function u₀ or v₀ may be the aforementioned unified feature, that is, the surface feature function of the molecule. In this way, embodiments of the present disclosure can obtain the time-dependent evolution multiscale feature, that is, uₜ or vₜ, based on the time-dependent evolution operator.

Exemplarily, the time-dependent evolution operator e^{-Δt} in formula (7) may be referred to as a heat operator, which describes the distribution vₜ of the initial heat distribution v₀ in the manifold space after time t.

As an example, FIG. 5 illustrates a schematic diagram of a change in heat distribution on the molecular surface over time. It may be understood that this change can be quantitatively described by the time-dependent evolution process shown in formula (7).

As can be seen from FIG. 5, as the time t becomes larger and larger, the range of heat transfer becomes farther and farther. Therefore, by controlling different evolution times t, multi-scale information transfer (short time corresponds to small-scale information transfer, and long time corresponds to large-scale information transfer) can be implemented in the Riemannian manifold space of the molecular surface. Therefore, the geometric and chemical information of the molecule at different scales can be learned by using the time-dependent evolution-based neural network, thereby improving the representation ability for the molecule.

In embodiments of the present disclosure, the eigenfunction and the eigenvalue of the Laplace operator are described above in conjunction with formula (1). Thus, the time-dependent evolution operator may be based on the eigenfunction and the eigenvalue of the Laplace operator on the Riemannian manifold. Based on this, formula (7) may be further expressed as the following formula (8):

$${t}_{} = {e}^{- Δ t} {0}_{} = Φ [ \begin{matrix}{e}^{- {λ}_{0} t} \\ {e}^{- {λ}_{1} t} \\ ⋮\end{matrix} ] ⊙ ( {Φ}^{T} {0}_{} ) \qquad ( 8 )$$

Similarly, formula (6) may be further expressed as the following formula (9):

$${u}_{t} = {e}^{- i Δ t} {u}_{0} = Φ [ \begin{matrix}{e}^{- i {λ}_{0} t} \\ {e}^{- i {λ}_{1} t} \\ ⋮\end{matrix} ] ⊙ ( {Φ}^{T} {u}_{0} ) \qquad ( 9 )$$

In this way, embodiments of the present disclosure can perform time-dependent evolution in the eigen space by using the Riemannian manifold and the eigenfunction and the eigenvalue of its Laplace operator, which is more efficient than the operation in the real space.

As mentioned above, the unified feature may be represented as, for example, a 64-dimensional surface feature function, that is, each node of the molecular surface may be represented by a 64-dimensional array for the unified feature of the node. Then, based on formula (8) or formula (9), the time-dependent evolution may be performed on the functions of the 64 dimensions respectively. It may be understood that each function may have its unique evolution time, for example, t may be used as a parameter for the neural network for the time-dependent evolution or may be set by the user. After the time-dependent evolution, the multiscale feature on the molecular surface can be obtained, including a series of geometric and chemical features at different scales.

In embodiments of the present disclosure, the time-dependent evolution multiscale feature provides a molecule representation method based on the Riemannian manifold, which is different from the existing molecule representation methods. The time-dependent evolution multiscale feature includes the geometric feature and the chemical feature of the molecule, which enhances the description ability of the molecule feature.

With continued reference to FIG. 1, at block 110, the binding site may be determined based on the time-dependent evolution multiscale feature. It may be understood that the binding site is a region on the molecule, which can bind to another molecule.

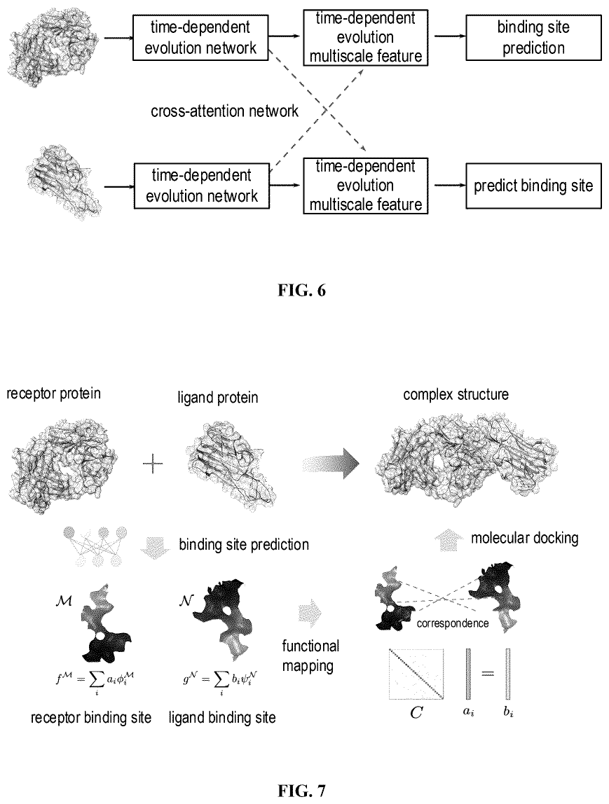

Additionally or alternatively, a cross-attention network may be further used. It may be understood that the information exchange between the two molecules may be implemented through the cross-attention network. For example, for each node on the first molecular surface of the first molecule, the attention to each node on the second molecular surface of the second molecule may be calculated respectively, where the attention may be the inner product of the features of two different nodes, and the attention may reflect the “correlation” between the two different nodes. Subsequently, the attention may be normalized, and the features of the nodes may be updated in a crossed manner, for example, the feature of the node on the first molecular surface is used to update the feature of the node on the second molecular surface, and the feature of the node on the second molecular surface is used to update the feature of the node on the first molecular surface. FIG. 6 illustrates a schematic diagram of using a cross-attention network. With reference to FIG. 6, the time-dependent evolution multiscale features of the two molecules may be respectively obtained through the combination of the time-dependent evolution neural network and the cross-attention network. Furthermore, the time-dependent evolution multiscale features can be used for the subsequent prediction of the binding site.

Exemplarily, assuming that the first molecule is a receptor protein and the second molecule is a ligand protein, at least one node in a plurality of surface nodes on the molecular surface of the second molecule may be determined based on the second time-dependent evolution multiscale feature of the second molecule, and the at least one node indicates a site for binding to the first molecule. For example, a first region (such as part or all region of the molecule surface) of the molecular surface of the second molecule may be obtained, and for each surface node in the first region, it may be analyzed whether each node can bind to the first molecule, so as to achieve the binary prediction.

In some examples, the first binding site is a sub-region of the first molecular surface of the first molecule (for example, referred to as a first sub-region), and the second binding site is another sub-region of the second molecular surface of the second molecule (for example, referred to as a second sub-region). Exemplarily, the first sub-region may be represented as a Riemannian manifold [symbol] , and the second sub-region may be represented as a Riemannian manifold [symbol] .

As described above in the process of describing the time-dependent evolution multiscale feature of the molecule, the surface chemical feature of the molecule may be obtained. Accordingly, it may be understood that at block 120, the first chemical feature may be obtained based on the first surface chemical feature of the first molecule based on the surface nodes included in the first sub-region. The second chemical feature may be obtained based on the second surface chemical feature of the second molecule based on the surface nodes included in the second sub-region.

In embodiments of the present disclosure, the first chemical feature and the second chemical feature have the same attribute, e.g., which may be an electrostatic potential energy function.

In some examples, the chemical feature may be represented as a linear combination of the corresponding eigenfunctions. For example, the first chemical feature is represented as a linear combination of the eigenfunctions of the Laplace operator on the Riemannian manifold of the first sub-region, and the second chemical feature is represented as a linear combination of the eigenfunctions of the Laplace operator on the Riemannian manifold of the second sub-region. For the purpose of a simplified description, the first chemical feature and the second chemical feature may be represented as:

$${f}^{ℳ} = {\sum }_{i} , = {\sum }_{i} \qquad ( 10 )$$

In formula (10), [symbol] represents the eigenfunction of the Laplace operator on the Riemannian manifold of the first sub-region, [symbol] represents the eigenfunction of the Laplace operator on the Riemannian manifold of the second sub-region, and aᵢ and bᵢ are respectively coefficients of the linear combination.

Based on the first chemical feature and the second chemical feature, the first coefficient matrix (for example, represented as A) and the second coefficient matrix (for example, represented as B) may be determined accordingly. Further, the functional mapping matrix may be determined based on the first coefficient matrix and the second coefficient matrix.

For example, the functional mapping matrix (such as C) may be represented as

$${\sum }_{i} {i}_{} {c}_{i j} = {b}_{} \qquad ( 11 )$$

$$or$$

$$CA = B \qquad ( 12 )$$

Exemplarily, the functional mapping matrix may be a kernel function, and the correspondence between the first sub-region and the second sub-region may be determined through the functional mapping matrix. In other words, it may be determined which node in the second sub-region is corresponding to each node in the first sub-region. For example, for a certain node in the first sub-region, the position of the corresponding node in the second sub-region may be determined.

In some examples, at block 150, the correspondence may be transformed into a corresponding translation operation and a rotation operation through a geometric algorithm, thereby realizing the docking. For example, the ligand protein is docked onto the receptor protein. Therefore, the structure of the protein complex can be accurately predicted.

Taking the first molecule as a receptor protein and the second molecule as a ligand protein as an example, FIG. 7 illustrates a schematic diagram of molecular docking in accordance with embodiments of the present disclosure.

It may be understood that although the embodiment of the docking is described in the process 100 with reference to the first molecule and the second molecule, embodiments of the present disclosure may be applied to a larger number of molecules. For example, if the first molecule is a virus, proteins that may have the possibility of docking to the virus may be screened from a large number of known protein structure databases. Optionally, the binding region of the selected protein may be further optimized based on the binding site, so that the protein has a stronger binding ability to the virus, so that an effective antibody drug can be obtained more quickly.

It should be understood that in embodiments of the present disclosure, “first”, “second”, “third”, etc. are only used to indicate that a plurality of objects may be different, but at the same time, the possibility that two objects are the same is not excluded, and it should not be construed as any limitation to embodiments of the present disclosure.

It should also be understood that the manners, situations, categories and the division of the embodiments in the embodiments of the present disclosure are only for the convenience of description, and should not constitute a specific limitation. The features in various manners, categories, situations and embodiments may be combined with each other under a logical condition.

It should also be understood that the above content is only to help those skilled in the art better understand the embodiments of the present disclosure, and is not intended to limit the scope of the embodiments of the present disclosure. Those skilled in the art can make various modifications, changes or combinations, etc. according to the above content. Such a modified, changed or combined solution is also within the scope of the embodiments of the present disclosure.

It should also be understood that the description of the above content focuses on emphasizing the differences between the embodiments, and the same or similar parts may refer to each other or draw lessons from each other. For the sake of brevity, it will not be repeated here.

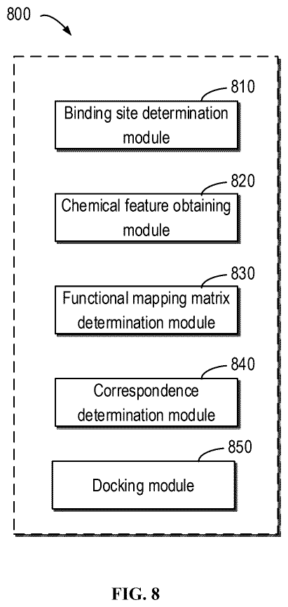

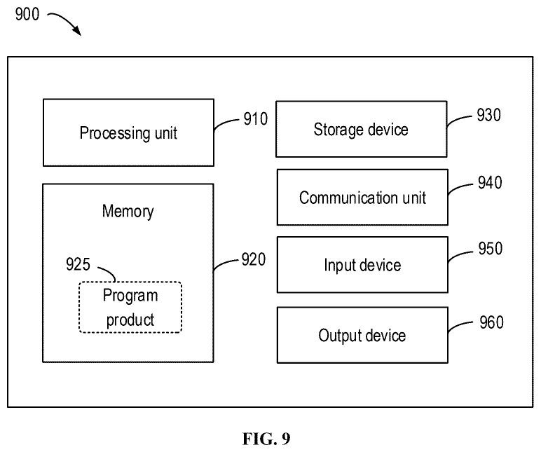

FIG. 8 illustrates a schematic block diagram of an example apparatus 800 in accordance with some embodiments of the present disclosure. The apparatus 800 may be implemented by means of software, hardware or a combination of both. As shown in FIG. 8, the apparatus 800 includes a binding site determination module 810, a chemical feature obtaining module 820, functional mapping matrix determination module 830, a correspondence determination module 840, and a docking module 850.

The binding site determination module 810 is configured to determine a first binding site on a first molecular surface of a first molecule and a second binding site on a second molecular surface of a second molecule based on a first time-dependent evolution multiscale feature of the first molecule and a second time-dependent evolution multiscale feature of the second molecule. The chemical feature obtaining module 820 is configured to obtain a first chemical feature of the first binding site and a second chemical feature of the second binding site. The functional mapping matrix determination module 830 is configured to determine a functional mapping matrix between the first chemical feature and the second chemical feature through functional mapping. The correspondence determination module 840 is configured to determine a correspondence between the first binding site and the second binding site based on the functional mapping matrix. The docking module 850 is configured to dock the first molecule and the second molecule through the first binding site and the second binding site based on the correspondence.

In some embodiments, the apparatus 800 further includes: a surface node determination module, configured to determine a first molecular surface of the first molecule, where the first molecular surface is a continuous Riemannian manifold and the first molecular surface includes a plurality of discrete surface nodes; a geometric feature determination module, configured to determine a first geometric feature of the first molecule based on the first molecular surface; a surface chemical feature determination module, configured to determine a first surface chemical feature of the first molecule by mapping atomic information inside the first molecule to the plurality of surface nodes; and a time-dependent evolution multiscale feature determination module, configured to determine the first time-dependent evolution multiscale feature of the first molecule based on the first geometric feature and the first surface chemical feature.

Exemplarily, the surface node determination module is configured to determine the first molecular surface based on an isosurface of an electron density field of the first molecule. Alternatively, the surface node determination module is configured to determine the first molecular surface based on the sampling of the solvent-accessible surface or the solvent-inaccessible surface of the first molecule.

Optionally, the first geometric feature includes at least one of the following: a heat kernel signature determined based on the eigenfunction and the eigenvalue of the Laplace operator on the first molecular surface, a wave kernel signature determined based on the eigenfunction and the eigenvalue of the Laplace operator on the first molecular surface. Gaussian curvature of the first molecular surface, or mean curvature of the first molecular surface.

Exemplarily, the surface chemical feature determination module includes: a chemical environment feature determination sub-module, configured to obtain a chemical environment feature of the node by mapping atomic information of a plurality of atoms associated with the node to the node for each node in the plurality of surface nodes; and a surface chemical feature determination sub-module, configured to determine the first surface chemical feature using a fully connected neural network based on the chemical environment feature of each node in the plurality of surface nodes.

Exemplarily, the time-dependent evolution multiscale feature determination module is configured to determine the unified feature of the first molecule by integrating the first geometric feature and the first surface chemical feature; and determine the first time-dependent evolution multiscale feature based on the unified feature based on a time-dependent evolution neural network model.

Optionally, the time-dependent evolution neural network model includes an evolution operator, and the evolution operator is determined based on at least one of the following: an eigenfunction of a Laplace operator on a Riemannian manifold, or a surface potential energy term, where the surface potential energy term is a function distribution on the Riemannian manifold set by the user.

In some embodiments, the binding site determination module 810 may be configured to determine the first binding site and the second binding site by using a cross-attention network.

Exemplarily, the first chemical feature is represented as a linear combination of the eigenfunctions of the Laplace operator on the Riemannian manifold of the first binding site, and the second chemical feature is represented as a linear combination of the eigenfunctions of the Laplace operator on the Riemannian manifold of the second binding site.

In some embodiments, the functional mapping matrix determination module 830 is configured to determine a first coefficient matrix of the first chemical feature; determine a second coefficient matrix of the second chemical feature; and determine the functional mapping matrix based on the first coefficient matrix and the second coefficient matrix.

In some embodiments, the apparatus 800 may further include a complex structure determination module, configured to determine a structure of the complex after the first molecule and the second molecule are docked based on the docking between the first binding site and the second binding site.

The apparatus 800 of FIG. 8 can be used to implement the process described above with reference to FIGS. 1-7. For the sake of brevity, it will not be repeated here.

The division of the modules or units in the embodiments of the present disclosure is schematic, and is only a logical function division. There may be another division manner in actual implementation. In addition, the functional units in the embodiments of the present disclosure may be integrated into one unit, or may exist separately physically, or two or more units may be integrated into one unit. The above integrated unit may be implemented in the form of hardware, or may be implemented in the form of a software functional unit.

FIG. 9 illustrates a block diagram of an example device 900 that may be used to implement the embodiments of the present disclosure. It should be understood that the device 900 shown in FIG. 9 is merely an example, and should not impose any limitation on the function and scope of the implementations described herein. For example, the device 900 may be used to perform the process described above with reference to FIGS. 1-7. For example, the device 900 may be implemented as a classical computer and/or a quantum computer.

As shown in FIG. 9, the device 900 is in the form of a general-purpose computing device. The components of the computing device 900 may include, but are not limited to, one or more processors or processing units 910, a memory 920, a storage device 930, one or more communication units 940, one or more input devices 950, and one or more output devices 960. The processing unit 910 may be an actual or virtual processor and can perform various processes according to a program stored in the memory 920. In a multiprocessor system, multiple processing units execute computer-executable instructions in parallel to improve the parallel processing capability of the computing device 900.

The computing device 900 typically includes a plurality of computer storage media. Such media may be any available media accessible by the computing device 900, including but not limited to volatile and non-volatile media, removable and non-removable media. The memory 920 may be a volatile memory (for example. a register, a cache, a random access memory (RAM)), a non-volatile memory (for example, a read-only memory (ROM), an electrically erasable programmable read-only memory (EEPROM), a flash memory), or some combination thereof. The storage device 930 may be a removable or non-removable medium and may include a machine-readable medium, such as a flash drive, a disk, or any other medium, which may be used to store information and/or data (for example, training data for training) and may be accessed within the computing device 900.

The computing device 900 may further include additional removable/non-removable, volatile/non-volatile storage media. Although not shown in FIG. 9, a disk drive for reading from or writing to a removable, non-volatile disk (for example, a “floppy disk”) and an optical disk drive for reading from or writing to a removable, non-volatile optical disk may be provided. In these cases, each drive may be connected to a bus (not shown) through one or more data medium interfaces. The memory 920 may include a computer program product 925, which has one or more program modules, which are configured to perform various methods or actions of various implementations of the present disclosure.

The communication unit 940 communicates with other computing devices through a communication medium. Additionally, the functions of the components of the computing device 900 may be implemented by a single computing cluster or multiple computing machines that can communicate through a communication connection. Therefore, the computing device 900 may operate in a networked environment using logical connections to one or more other servers, network personal computers (PCs), or another network node.

The input device 950 may be one or more input devices, such as a mouse, a keyboard, a trackball, etc. The output device 960 may be one or more output devices, such as a display, a speaker, a printer, etc. The computing device 900 may also communicate with one or more external devices (not shown) through the communication unit 940 as needed, such as a storage device, a display device, etc., communicate with one or more devices that enable a user to interact with the computing device 900, or communicate with any device (for example, a network card, a modem, etc.) that enables the computing device 900 to communicate with one or more other computing devices. Such communication may be performed via an input/output (I/O) interface (not shown).

According to an exemplary implementation of the present disclosure, a computer-readable storage medium having computer-executable instructions stored thereon is provided, where the computer-executable instructions, when executed by a processor, implement the method described above. According to an exemplary implementation of the present disclosure, a computer program product is further provided. The computer program product is tangibly stored on a non-transitory computer-readable medium and includes computer-executable instructions, and the computer-executable instructions, when executed by a processor, implement the method described above. According to an exemplary implementation of the present disclosure, a computer program product having a computer program stored thereon is provided. The program, when executed by a processor, implements the method described above.

Various aspects of the present disclosure are described herein with reference to the flowcharts and/or block diagrams of the methods, apparatus, devices and computer program products implemented according to the present disclosure. It should be understood that each block of the flowchart and/or block diagram and a combination of blocks in the flowchart and/or block diagram may be implemented by computer-readable program instructions.

These computer-readable program instructions may be provided to a processing unit of a general-purpose computer, a special-purpose computer, or another programmable data processing apparatus to produce a machine, such that these instructions, when executed by a processing unit of a computer or another programmable data processing apparatus, generate an apparatus for implementing the functions/acts specified in one or more blocks in the flowchart and/or block diagram. These computer-readable program instructions may also be stored in a computer-readable storage medium. These instructions enable a computer, a programmable data processing apparatus, and/or another device to work in a specific manner. Therefore, the computer-readable medium storing the instructions includes a product, which includes instructions for implementing various aspects of the functions/acts specified in one or more blocks in the flowchart and/or block diagram.

The computer-readable program instructions may be loaded onto a computer, another programmable data processing apparatus, or another device, so that a series of operation steps are performed on the computer, another programmable data processing apparatus, or another device to produce a computer-implemented process, such that the instructions executed on the computer, another programmable data processing apparatus, or another device implement the functions/acts specified in one or more blocks in the flowchart and/or block diagram.

The flowcharts and block diagrams in the accompanying drawings illustrate the possibly implemented architecture, functions and operations of the system, method and computer program product according to a plurality of implementations of the present disclosure. In this regard, each block in the flowchart or block diagram may represent a module, program segment or part of an instruction, and the module, program segment or part of an instruction contains one or more executable instructions for implementing the specified logical functions. In some alternative implementations, the functions marked in the blocks may also occur in an order different from that marked in the accompanying drawings. For example, two blocks shown in succession may actually be performed substantially in parallel, or they may sometimes be performed in a reverse order, depending on the function involved. It should also be noted that each block in the block diagram and/or the flowchart, and a combination of the blocks in the block diagram and/or the flowchart may be implemented by a dedicated hardware-based system that performs specified functions or acts, or may be implemented by a combination of dedicated hardware and computer instructions.

The foregoing has described various implementations of the present disclosure. The foregoing description is exemplary, not exhaustive, and is not limited to the disclosed implementations. Many modifications and changes are obvious to those of ordinary skill in the art without departing from the scope and spirit of the described implementations. The selection of terms used herein is intended to best explain the principles of the implementations, the practical application or the improvement to the technology in the market, or to enable other ordinary skilled persons in the art to understand the various implementations disclosed herein.

1. A method for molecular docking, comprising:
determining a first binding site on a first molecular surface of a first molecule and a second binding site on a second molecular surface of a second molecule based on a first time-dependent evolution multiscale feature of the first molecule and a second time-dependent evolution multiscale feature of the second molecule;
obtaining a first chemical feature of the first binding site and a second chemical feature of the second binding site;
determining a functional mapping matrix between the first chemical feature and the second chemical feature through functional mapping;
determining a correspondence between the first binding site and the second binding site based on the functional mapping matrix; and
docking the first molecule and the second molecule through the first binding site and the second binding site based on the correspondence.

2. The method of claim 1, further comprising:
determining the first molecular surface of the first molecule, wherein the first molecular surface is a continuous Riemannian manifold and the first molecular surface comprises a plurality of discrete surface nodes;
determining a first geometric feature of the first molecule based on the first molecular surface;
determining a first surface chemical feature of the first molecule by mapping atomic information inside the first molecule to the plurality of discrete surface nodes; and
determining the first time-dependent evolution multiscale feature of the first molecule based on the first geometric feature and the first surface chemical feature.

3. The method of claim 2, wherein determining the first molecular surface comprises:
determining the first molecular surface based on an isosurface of an electron density field of the first molecule; or
determining the first molecular surface based on sampling of solvent-accessible or solvent-inaccessible surfaces of the first molecule.

4. The method of claim 2, wherein the first geometric feature comprises at least one of:
a heat kernel signature determined based on an eigenfunction and an eigenvalue of a Laplace operator on the first molecular surface,
a wave kernel signature determined based on the eigenfunction and the eigenvalue of the Laplace operator on the first molecular surface,
Gaussian curvature of the first molecular surface, or
mean curvature of the first molecular surface.

5. The method of claim 2, wherein determining the first surface chemical feature comprises:
obtaining a chemical environment feature of a node by mapping atomic information of a plurality of atoms associated with the node to the node for each node in the plurality of discrete surface nodes; and
determining the first surface chemical feature using a fully connected neural network based on the chemical environment feature of each node in the plurality of discrete surface nodes.

6. The method of claim 2, wherein determining the first time-dependent evolution multiscale feature comprises:
determining a unified feature of the first molecule by integrating the first geometric feature and the first surface chemical feature; and
determining the first time-dependent evolution multiscale feature based on the unified feature using a time-dependent evolution neural network model.

7. The method of claim 6, wherein the time-dependent evolution neural network model comprises an evolution operator, and the evolution operator is determined based on at least one of:
an eigenfunction of a Laplace operator on a Riemannian manifold, or
a surface potential energy term, wherein the surface potential energy term is a function distribution on the Riemannian manifold set by a user.

8. The method of claim 1, wherein determining the first binding site and the second binding site comprises:
determining the first binding site and the second binding site by using a cross-attention network.

9. The method of claim 1, wherein the first chemical feature is represented as a linear combination of eigenfunctions of a Laplace operator on a Riemannian manifold of the first binding site, and the second chemical feature is represented as a linear combination of eigenfunctions of a Laplace operator on a Riemannian manifold of the second binding site.

10. The method of claim 1, wherein determining the functional mapping matrix between the first chemical feature and the second chemical feature comprises:
determining a first coefficient matrix of the first chemical feature;
determining a second coefficient matrix of the second chemical feature; and
determining the functional mapping matrix based on the first coefficient matrix and the second coefficient matrix.

11. The method of claim 1, further comprising:
determining a structure of a complex after the first molecule and the second molecule are docked based on the docking between the first binding site and the second binding site.

12. An electronic device, comprising:
at least one processing unit;
at least one memory, the at least one memory being coupled to the at least one processing unit and storing instructions executable by the at least one processing unit, the instructions, when executed by the at least one processing unit, causing the electronic device to perform actions, the actions comprising:
determining a first binding site on a first molecular surface of a first molecule and a second binding site on a second molecular surface of a second molecule based on a first time-dependent evolution multiscale feature of the first molecule and a second time-dependent evolution multiscale feature of the second molecule;
obtaining a first chemical feature of the first binding site and a second chemical feature of the second binding site;
determining a functional mapping matrix between the first chemical feature and the second chemical feature through functional mapping;
determining a correspondence between the first binding site and the second binding site based on the functional mapping matrix; and
docking the first molecule and the second molecule through the first binding site and the second binding site based on the correspondence.

13. (canceled)

14. A non-transitory computer-readable storage medium having a computer program stored thereon, the computer program, when executed by a processor, implementing a method comprising:
determining a first binding site on a first molecular surface of a first molecule and a second binding site on a second molecular surface of a second molecule based on a first time-dependent evolution multiscale feature of the first molecule and a second time-dependent evolution multiscale feature of the second molecule;
obtaining a first chemical feature of the first binding site and a second chemical feature of the second binding site;
determining a functional mapping matrix between the first chemical feature and the second chemical feature through functional mapping;
determining a correspondence between the first binding site and the second binding site based on the functional mapping matrix; and
docking the first molecule and the second molecule through the first binding site and the second binding site based on the correspondence.

15. The electronic device of claim 12, the actions further comprising:
determining the first molecular surface of the first molecule, wherein the first molecular surface is a continuous Riemannian manifold and the first molecular surface comprises a plurality of discrete surface nodes;
determining a first geometric feature of the first molecule based on the first molecular surface;
determining a first surface chemical feature of the first molecule by mapping atomic information inside the first molecule to the plurality of discrete surface nodes; and
determining the first time-dependent evolution multiscale feature of the first molecule based on the first geometric feature and the first surface chemical feature,
wherein the first geometric feature comprises at least one of:
a heat kernel signature determined based on an eigenfunction and an eigenvalue of a Laplace operator on the first molecular surface,
a wave kernel signature determined based on the eigenfunction and the eigenvalue of the Laplace operator on the first molecular surface,
Gaussian curvature of the first molecular surface, or
mean curvature of the first molecular surface.

16. The electronic device of claim 15, wherein determining the first molecular surface comprises:
determining the first molecular surface based on an isosurface of an electron density field of the first molecule; or
determining the first molecular surface based on sampling of solvent-accessible or solvent-inaccessible surfaces of the first molecule.

17. The electronic device of claim 15, wherein determining the first surface chemical feature comprises:
obtaining a chemical environment feature of a node by mapping atomic information of a plurality of atoms associated with the node to the node for each node in the plurality of discrete surface nodes; and
determining the first surface chemical feature using a fully connected neural network based on the chemical environment feature of each node in the plurality of discrete surface nodes.

18. The electronic device of claim 15, wherein determining the first time-dependent evolution multiscale feature comprises:
determining a unified feature of the first molecule by integrating the first geometric feature and the first surface chemical feature; and
determining the first time-dependent evolution multiscale feature based on the unified feature using a time-dependent evolution neural network model,
wherein the time-dependent evolution neural network model comprises an evolution operator, and the evolution operator is determined based on at least one of:
an eigenfunction of a Laplace operator on a Riemannian manifold, or
a surface potential energy term, wherein the surface potential energy term is a function distribution on the Riemannian manifold set by a user.

19. The electronic device of claim 12, wherein determining the first binding site and the second binding site comprises:
determining the first binding site and the second binding site by using a cross-attention network.

20. The electronic device of claim 12, wherein the first chemical feature is represented as a linear combination of eigenfunctions of a Laplace operator on a Riemannian manifold of the first binding site, and the second chemical feature is represented as a linear combination of eigenfunctions of a Laplace operator on a Riemannian manifold of the second binding site.

21. The electronic device of claim 12, wherein determining the functional mapping matrix between the first chemical feature and the second chemical feature comprises:
determining a first coefficient matrix of the first chemical feature;
determining a second coefficient matrix of the second chemical feature; and
determining the functional mapping matrix based on the first coefficient matrix and the second coefficient matrix.
